(19) United States
(12) Patent Application Publication
(10) Pub. No.: US 2011/0166101 A1
(43) Pub. Date: Jul. 7, 2011

(54) MICROPARTICLES

(75) Inventors: Subhas Balaram Bhowmick, Baroda (IN); Lalatendu Panigrahi, Baroda (IN); Sujit Kumar Dolai, Baroda (IN)

(73) Assignee: SUN PHARMA ADVANCED RESEARCH COMPANY LIMITED, Mumbai (IN)

(21) Appl. No.: 13/061,316

(22) PCT Filed: Aug. 26, 2009
(86) PCT No.: PCT/IN2009/000469

§ 371 Date: Feb. 28, 2011

(30) Foreign Application Priority Data
Aug. 29, 2008 (IN) 1820/MUM/2008

(51) Int. Cl. A61K 31/715

(52) U.S. Cl. 514/57

(57) ABSTRACT

A substantially porous, micro-particle comprising therapeutically effective amounts of tretinoin and ethyl cellulose

### FIELD OF THE INVENTION

The present invention relates to microparticles of tretinoin.

### BACKGROUND OF THE INVENTION

Tretinoin, chemically termed as all-trans-retinoic acid, also known as (all-E)-3,7-dimethyl-9-(2,6,6-trimethyl-1-cyclohexen-1-yl)-2-4,6,8-nonatetraenoic acid is the most commonly used drug in the treatment of Acne vulgaris. It is commercially available in United States of America in the form of creams, gels and solutions for topical administration. Most of these compositions release the active agents rapidly, resulting in the need for repeated application. Controlled release compositions have been developed to overcome the problems of repeated application requirements. One of such compositions approved in the United States of America is Retin-A MICRO® topical gel for controlled delivery of tretinoin.

The marketed product namely, Retin-A MICRO® (Tretinoin gel) is a microsphere formulation containing 0.1% or 0.04%, by weight. This formulation is a gel based vehicle comprised of polymeric beads having a network of pores with the active ingredient held within the network to provide a controlled time release of the active ingredient. Such polymeric beads may be incorporated in a medium, such as a gel, a cream, a lotion, an ointment, a liquid or the like which may be applied to a surface. The active ingredient may then be released by pressure, diffusion or volatilization. The delivery vehicle has increased mechanical stability over a microencapsulated or gel delivery vehicle. The network of pores of a bead would not get subjected to osmotic shock allowing easy handling during manufacture. Several such polymeric beads are disclosed in the prior art for example, U.S. Pat. No. 5,145,675; U.S. Pat. No. 4,690,825 and U.S. Pat. No. 5,955,109 (“the 109 patent”).

The U.S. Pat. No. 5,955,109 discloses a topical composition for delivery of retinoic acid to the skin, the composition comprising:
(a) solid particles composed of a cross linked copolymer of monoethylenically unsaturated monomers and polyethylenically unsaturated monomers free from reactive functionalities and having a cross-linking density from 20% to 80%, wherein said particles contain a continuous collapsible network of pores open to the exterior of said particles, are spherical in shape, and have an average diameter of 1 micron to about 100 microns, a total pore volume of about 0.01 cc/g to about 40 cc/g, a surface area of about 1 m²/g to about 500 m²/g, and an average pore diameter of about 0.001 micron to about 3.0 microns, and
(b) an impregnant comprising tretinoin acid retained inside said pores in an amount effective to promote skin repair, wherein retention of said retinoic acid inside said pores reduces irritancy of the composition when compared to application of the same amount of free retinoic acid without loss of skin repair promotion activity.

The pores thus formed according to the U.S. Pat. No. '109 are interconnected and open to the particle surface, permitting full diffusion outward of the retained retinoic acid under particular conditions. Although the '109 patent discloses an effective means for sustaining the release, it provides a two step process of making the microparticles which is very tedious. The process disclosed in U.S. Pat. No. '109 requires a separate process of polymerization of the monomers and a use of porogen to form the pores. The retinoid impregnant may be placed inside the pores of preformed dry porous polymer beads. Moreover, the cross-linking in the polymer formation is a major means of pore size control. The patent teaches to use copolymerization of styrene and divnylbenzene, vinyl stearate and divinylbenzene, 4-vinyl pyridine and ethylene glycol dimethacrylate which are all synthetic polymers. Such polymers may not be always a choice especially in comparison to the polymers of natural origin, for example, semi-synthetic polymers such as ethyl cellulose. It is desirable to utilize a naturally occurring or chemically modified natural polymers for example, cellulose derivatives such as ethyl cellulose instead of the synthetic polymers.

Use of ethyl cellulose has been investigated in PCT Publication Number WO2006/133131 (hereinafter referred to as PCT publication '131). This application discloses use of substantially non-porous polymeric microparticles comprising a hydrophobic polymer and a plasticizer, and containing therein a bioactive or inactive agent. Although PCT publication '131 discloses use of ethyl cellulose as the polymer for the microparticles, the microparticles are substantially non porous and the pore diameter of particles is in the range of few nanometers to about one micron in size with a total pore volume of about 0.000552 cm³/g. The '131 patent publication discloses use of about 0.5% of polyvinyl alcohol as a suspending agent. It has been found by the inventors of the present invention that such high amount of polyvinyl alcohol leads to several process problems such as foaming, clogging of the filter. Also the use of higher amount of a suspending agent as disclosed in PCT publication '131 leads to undue delay in the whole process of making the microparticles. Also, the PCT publication '131 discloses the use of plasticizers during the preparation of microparticles wherein the average pore diameter was about 1 micron or less.

U.S. Pat. No. 5,725,869 claims a process for producing porous spongy microspheres characterized by an uneven porous surface and a porous internal structure resembling a sponge having a diameter between about 3 to about 300 microns comprising:
(a) preparing an organic phase comprising a solution of a polymer and a plasticizer selected from the group consisting of phthalate esters, phosphate esters, citrate, sebacate esters, glycerol, triacetin and acetylated, monoglycerides in an organic solvent;
(b) preparing an aqueous phase comprising an aqueous solution of one or more emulsifying agents;
(c) combining the organic and aqueous phases under emulsifying conditions to form an emulsion of the organic phase in the aqueous phase; and (d) evaporating the solvent to form said porous, spongy microparticles of uneven porous surface and a porous internal structure resembling a sponge.

The patent teaches use of a plasticizer while preparing the microparticles. However, the inventors have observed that during the manufacture of microparticles using plasticizer there are little chances of plasticizer retaining in the finished microparticles due to high concentration of water present in the dispersed phase and during washing the plasticizer will come out from the microparticles. On use of higher quantity of plasticizers the finished microparticles may stick to each other causing agglomeration and poor yields. The inventors have found that the microparticles when manufactured without the use of plasticizer provided substantially porous microparticles with good yield up to 90%.

In an attempt to make environmental friendly microparticles prepared by using naturally occurring polymers, we have found that the tretinoin can be incorporated into the microparticles formed by use of ethyl cellulose, the said microparticles releasing the tretinoin in a sustained manner. When such particles are incorporated in a conventional vehicle such as for example, gel, the formulation is also found to be non irritant to the skin and comparable in terms of efficacy in treating the skin conditions such as Acne with the commercially available microparticle preparation namely, Retin A MICRO®.

### OBJECTS OF THE PRESENT INVENTION

It is the object of the present invention to provide microparticles comprising a polymer of natural or semi-synthetic origin.

It is yet another object of the invention to provide substantially porous microparticles with a pore size such that it ensures a desired sustained or controlled release of tretinoic acid upon topical application.

It is the object of the invention to provide a method of preparing substantially porous microparticles which is feasible for scale up till a batch size of about 5-10 kgs which requires very less time.

It is yet another object of the invention to provide a method which can be used for reproducibly producing in a substantially porous, spherical microparticles containing tretinoin.

### SUMMARY OF THE INVENTION

The present invention provides substantially porous, micro-particle comprising therapeutically effective amounts of tretinoin and ethyl cellulose.

The present invention, preferably provides substantially porous micro-particles wherein the micro-particles are free of plasticizers.

The present invention also provides substantially porous micro-particles wherein the average pore diameter of the micro-particles ranges from about 2 microns to 20 microns.

The present invention also provides a substantially porous, micro-particle wherein the ratio of ethyl cellulose to tretinoin ranges from about 99.0:1.0 to about 50:50.

The present invention further provides a substantially porous, micro-particle wherein tretinoin is loaded to about 1% by weight.

The present invention provides a substantially, porous micro-particle wherein the pores are formed in situ by use of volatile solvent such as dichloromethane.

In another aspect of the invention, the present invention also provides a process of preparing the substantially porous micro-particle which involves the steps of
i) dissolving tretinoin and ethyl cellulose in the organic solvent such as dichloromethane
ii) preparing an aqueous phase comprising a suspending agent
iii) adding the solution of step i) to solution of step ii under stirring or using homogenizer
iv) removing the organic solvent by stirring the emulsion, optionally, under vacuum, at a speed suitable to form the microparticles of desired porosity.

Also the present invention provides a process that does not require use of plasticizers.

The present invention may be summarized as follows:

A. A substantially porous, micro-particle comprising therapeutically effective amounts of tretinoin and ethyl cellulose.
B. A substantially porous, micro-particle as described in A above wherein the ratio of ethyl cellulose to tretinoin ranges from about 99.0:1.0 to about 50:50.
C. A substantially porous, micro-particle as described in A above wherein tretinoin is loaded to about 1% by weight.
D. A substantially, porous micro-particle as described in A above wherein the pores are formed in situ by use of volatile solvent such as dichloromethane.
E. A process of preparing the substantially porous micro-particle which involves the steps of
i) dissolving tretinoin and ethyl cellulose in the organic solvent such as dichloromethane
ii) preparing an aqueous phase comprising a suspending agent
iii) adding the solution of step i) to solution of step ii under stirring or using homogenizer
iv) removing the organic solvent by stirring the emulsion, optionally, under vacuum, at a speed suitable to form the microparticles of desired porosity.

### DESCRIPTION OF THE DRAWINGS AND FIGURES

FIG. 1 describes the images of the scanning electron microscopic view of the microparticles prepared according to example 1.

FIG. 1 describes the images of the scanning electron microscopic view of the microparticles prepared according to example 2.

FIG. 1 describes the images of the scanning electron microscopic view of the microparticles prepared according to example 3.

### DETAILED DESCRIPTION OF THE INVENTION

The present invention provides a substantially porous micro-particle comprising therapeutically effective amounts of tretinoin and ethyl cellulose.

The tretinoin is a term used for all-trans-forms of retinoic acid but includes other acids within the class such as the 9,10-cis-form and the 13-cis-form.

The term “microparticle” includes “microsphere” and the terms are used interchangeably in the present invention.

According to an embodiment of the invention, the microparticles contain tretinoin or its pharmaceutically acceptable salts. The amount of tretinoin present in the microparticles ranges from about 0.01% w/w to about 10.0% w/w, preferably, from about 0.05% w/w to about 5% w/w, more preferably from about 0.1% w/w to about 2% w/w.

The present invention provides the microparticles that are substantially porous in nature. In one embodiment of the present invention, the ‘substantially porous’ microparticles have an average pore diameter above 2 microns, preferably above 5 microns and most preferably ranging from about 12 microns to about 20 microns. The pore diameter and total porosity is determined by mercury dilatometer but any other suitable method may be employed.

According to the present invention, the average particle size of the microparticles ranges from about 15 microns to about 80 microns, preferably 25 microns to about 75 microns. The term average particle size as used herein means the mean particle size. The sizes of the microparticles can be determined using conventional methods of measuring and expressing particle size like Malvern particle size analysis, sieving, light scattering optical microscopy, image analysis, sedimentation and such other methods known to one skilled in the art. Particle size distribution information can be obtained from the values D₁₀, D₅₀, and D₉₀, such as can be generated from a Malvern particle size determination. D₉₀ as used herein is defined as the size for which 90 volume percent of the particles are smaller than that size given, and D₅₀ as used herein is defined as the size for which 50 volume percent of the particles are smaller than that size given. Likewise, D₁₀ as used herein is defined as the size for which 10 volume percent of the particles are smaller than that size given. The D90 of the microparticles ranges from about 30 microns to about 70 microns. In one embodiment, the microparticles of the present invention have a particle size of D₅₀<23.353 and D₉₀<53.798 and a specific surface area of about 0.425 m²/gm.

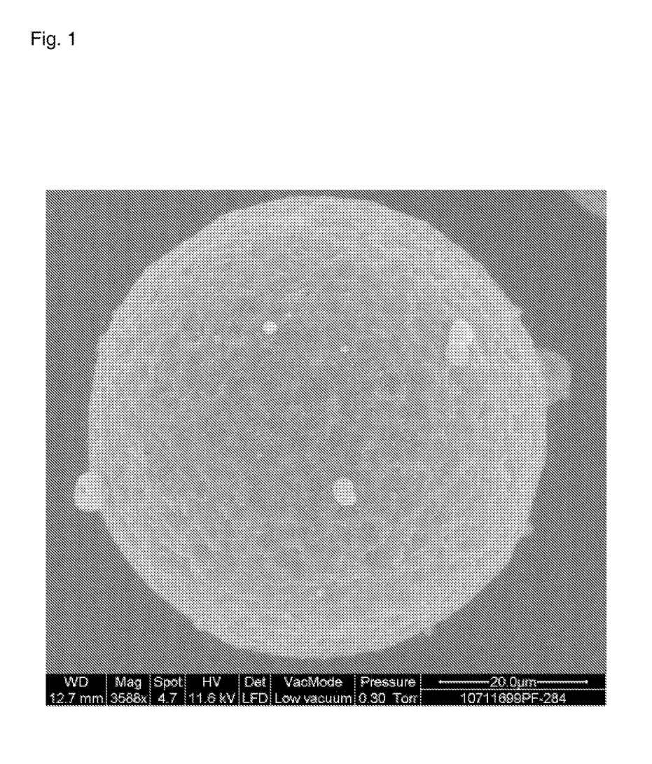

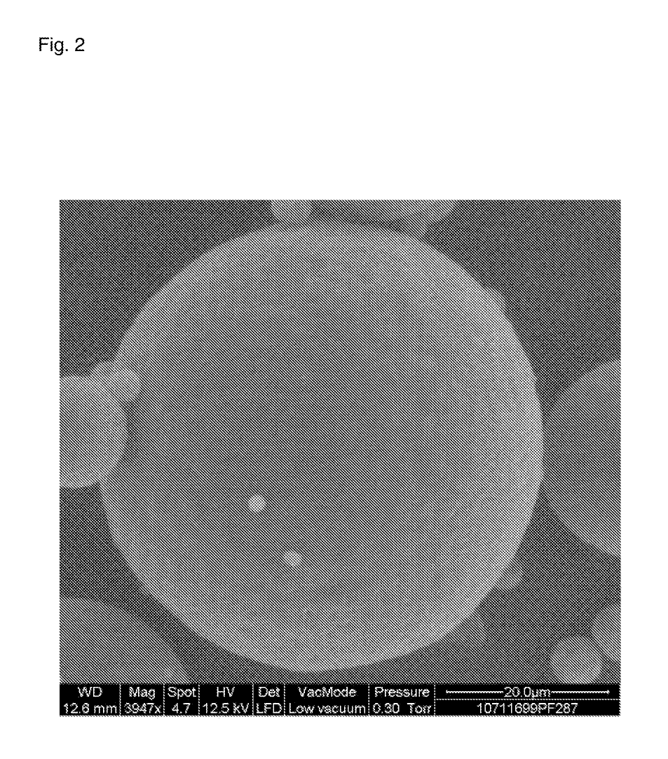

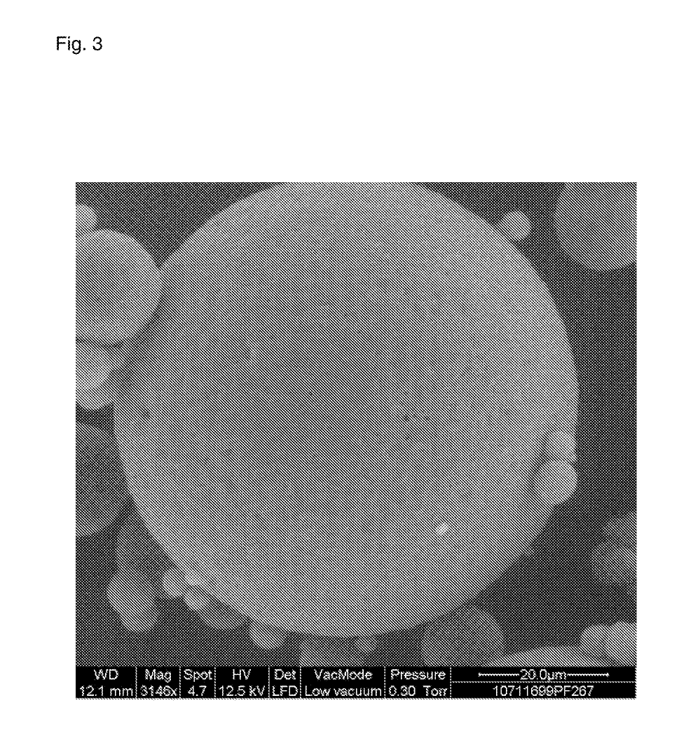

The microparticles according to the present invention may be of any shape including spherical, oblong and ellipsoidal and the like. In one embodiment of the present invention, the microparticles are substantially spherical in nature. FIG. 1 to FIG. 3 shows the SEM (scanning electron microscope) images of the microparticles prepared according to various embodiments of the present invention. The SEM images indicate the spherical and substantial porous nature of the microparticles. In one embodiment, without wishing to be bound by any theory, the inventors believe that because of the substantially uniform, porous spherical nature of the particles, the release of the drug from the microparticles incorporated in a suitable vehicle will be more uniform. Further the release of the drug from such compositions may be controlled by changing the size of the microparticles and its surface area. The specific surface area may be determined by any suitable method, for example, Malvern light scattering particle size measurement, BET and the like.

The present invention provides substantially porous, micro-particle comprising therapeutically effective amounts of tretinoin and ethyl cellulose. Preferably, the present invention provides substantially porous micro-particles wherein the micro-particles are free of plasticizers. Generally, the microparticles of the present invention have an average pore diameter of the micro-particles ranges from about 2 microns to 20 microns. The present invention provides a substantially, porous micro-particle wherein the pores are formed in situ by use of volatile solvent such as dichloromethane.

According to present invention, ethyl cellulose is used as the polymer for the microparticles. Ethyl cellulose has the polymeric “back bone” of cellulose, which is a naturally occurring polymer. The molecule has a structure of repeating anhydroglucose units. Ethyl cellulose is produced and marketed in a number of viscosity grades. Most commonly used grade of ethyl cellulose is commercially available from Dow chemical company Ltd., U.S.A, sold under the trade name ETHOCEL®. ETHOCEL polymers are produced in two types (standard and medium) that cover the range of the most useful ethoxyl content. “Standard” polymers have an ethoxy content of 48.0 to 49.5%; and “Medium” polymers have an ethoxyl content of 45.0 to 47.0%. Standard and Medium ethoxy types are available in premium grades, useful in regulated applications, and industrial grades. Premium grades are designed to meet the requirements of pharmaceutical applications. Examples of ethyl cellulose grades that can be used in the microparticles of the present invention include ethyl cellulose having a viscosity of about 4 cps to about 350 cps. Preferably, ethyl cellulose having a viscosity in the range of about 4 cps to about 100 cps is used. Most preferable, grades that can be used include, but are not limited to, ETHOCEL Std.4 PREMIUM, ETHOCEL Std.7 PREMIUM, ETHOCEL Std.10 PREMIUM, ETHOCEL Std.14 PREMIUM, ETHOCEL Std.20 PREMIUM, ETHOCEL Std.45, ETHOCEL Std. 100, ETHOCEL Std.200, and ETHOCEL Std. 300. Ethyl cellulose polymer can be used alone or in combination with two more grades of ethyl cellulose for effectively modulating the release of tretinoin from the microparticles when incorporated in a delivery vehicle. Preferably, the ethyl cellulose in the microparticle of the present invention is used in amount ranging from about 30% w/w to about 99% w/w, more preferably, about 50% to about 98% w/w, most preferably about 70% w/w to about 97% w/w.

The ratio of ethyl cellulose to tretinoin in the microparticles of the present invention can be varied to achieve a controlled release of tretinoin. In one embodiment of the present invention, the ratio of ethyl cellulose to tretinoin varies from about 50:1 to about 99:1, preferably varies from about 75:1 to about 95:1.

The microparticles of the present invention can further include suitable additives like antioxidants, preservatives known in the art. Preferably, butylated hydroxyl toluene is used as the antioxidant and is present in amounts ranging from about 0.01% w/w to about 5% w/w of the microparticles.

In one embodiment of the present invention, the microparticles comprises tretinoin, ethyl-cellulose and butylated hydroxyl toluene. More preferably, the micro-particles comprises from about 0.1% to about 2% tretinoin, about 50% to about 98% ethyl cellulose, and about 0.01% to about 5% butylated hydroxyl toluene, wherein the percentages are by weight of the microparticles.

It has been observed by the inventors of the present patent application that during the manufacture of microparticles if plasticizers are used, there are little chances of plasticizer retaining in the finished microparticles due to high concentration of water present in the dispersed phase and during washing the plasticizer will leach out from the microparticles. On use of higher quantity of plasticizers the finished microparticles may stick to each other causing agglomeration and poor yields. The inventors have found that the microparticles when manufactured without the use of plasticizer provided substantially porous microparticles with very good yield up to 90% and the process of manufacturing was industrially feasible, i.e microparticles could be manufactured at batch sizes of about 5-10 kgs efficiently with optimum utilization of time and energy.

According to the present invention, the novel microparticles of tretinoin may be incorporated in a delivery vehicle. The delivery vehicle that can be used to disperse the microparticles of tretinoin for topical administration should be biocompatible and should not cause any undue irritation to the application site. The delivery vehicle should be immiscible with the dispersed microparticles and have excellent spreadability. The vehicles may be in the form of gel, ointment, cream, paste and the like. The amount of microparticles in the delivery vehicle may vary from about 1% w/w to about 20% w/w, more preferably from about 5.0% w/w to about 15.0% w/w of the total delivery vehicle composition.

It is to be noted that the particle size of microparticles is important in terms of achieving the desired controlled release of tretinoin from the microparticles as well as in terms of spreadability, adhesion and feel when incorporated in the topical vehicle and applied on the skin. In one embodiment of the present invention, the inventors have found that the microparticles prepared according to the present invention with a D90 of the microparticles ranging from about 30 microns to about 70 microns have shown satisfactory results in terms of the physical appearance and the cosmetic effects such as feel after the topical application apart from achieving a control on the tretinoin release.

Examples of the delivery vehicle in which the microparticles of the present invention may be incorporated include, but are not limited to, acrylate polymers such as carbopols, carboxyvinyl polymers, xanthan gum, chitosan, povidone, polyethylene oxide, poloxamers, bentonite, methylcellulose, hydroxypropyl cellulose, hydroxypropyl methylcellulose alone or in combinations thereof. Preferable gelling agent that can be used in the composition of the present invention include acrylate polymers such as carbomer. Carbomer is commonly called as carbopol. Carbomer is a synthetic high molecular weight polymer of acrylic acid that is crosslinked with either allylsucrose or allyl ethers of pentaerythritol. It contains between 56-68% of carboxylic acid (—COOH) groups as calculated on the dry basis. Carbomers which are commercially available in various grades for use in delivery vehicles include but are not limited to carbomer 910, carbomer 934, carbomer 934P, carbomer 940, carbomer 941, carbomer 974P, carbomer 971P, carbomer 981, carbomer 1342 and mixtures thereof. Preferably, the amount of carbomer that may be used in the delivery vehicle ranges from about 0.01% w/w to about 10% w/w, more preferably about 0.1% w/w to about 8% w/w, most preferably 0.5% w/w to about 5% w/w of the total weight of the delivery vehicle comprising microparticles of the present invention. In one embodiment, the inventors have surprising found that when the microparticles were incorporated into an acrylic acid based gel, the release of the tretinoin was pH dependent.

Surfactants may be added to the delivery vehicle. Suitable surfactants that may be used in the composition of delivery vehicle of the present invention include PPG-20 methyl glucose distearate and cyclomethicone and dimethicone copolyol. PPG-20 methyl glucose distearate acts as a moisturizer or skin smoothening agent when used in the delivery vehicle and is present in amounts ranging from about 0.1% w/w to about 10.0% w/w, more preferably from about 1.0% w/w to about 7.0% w/w of the weight of the delivery vehicle. In one embodiment of the present invention, the delivery vehicle comprises Cyclomethicone and dimethicone copolyol as an emulsifying agent and is present in amounts ranging from about 0.1% w/w to about 10.0% w/w, preferably ranging from about 1.0% w/w to about 6.0% w/w.

The delivery vehicle may further include suitable additives like preservatives, antioxidants, opacifiers, emulsifiers, emollients, humectants, permeation enhancers, surfactants, chelating agents, pH regulators, stabilizers, hydrophilic fluids and other suitable pharmaceutically acceptable additives known in the art for preparation of a composition suitable for topical application.

Preservatives may be optionally incorporated into the delivery vehicle and the examples of such preservatives may include, but are not limited to, alkyl esters of para-hydroxybenzoic acid like methylparaben and propylparaben, benzoates, hydantoin derivatives, propionate salts, sorbic acid, benzyl alcohol, imidazolidinyl urea, sodium dehydroacetate and a variety of quaternary ammonium compounds. Preferably, preservatives can be used in amounts ranging from about 0.01% w/w to about 2% w/w of the microparticles.

The antioxidants which can be used in the delivery vehicle comprising the microparticles of the present invention should be non-reactive and should be safe for local use. Suitable antioxidants include butylated hydroxytoluene (BHT), butylated hydroxyanisole (BHA), ascorbic acid (vitamin C), propyl gallate, and alpha-tocopherol (vitamin E), although other antioxidants can be used provided. Preferably Butylated Hydroxy Toluene is used as the antioxidant and is used in amounts ranging from about 0.01% w/w to about 5% w/w of the delivery vehicle composition.

The hydrophilic fluids that can be used for the delivery vehicle include water, glycerol, propylene glycol, sorbitol and other higher alcohols and their mixtures in different proportions.

A pH regulator, normally a neutralizant agent, which can optionally have crosslinking function e.g. a ternary amine such as triethanolamine or trolamine, tromethamine, tetrahydroxypropylethylendiamine, etc; NaOH solution, etc may be added to the delivery vehicle. The preferred pH regulator is trolamine and present in the delivery vehicle in amounts ranging from about 0.05% w/w to about 2.0% w/w. The pH of the delivery vehicle may be adjusted to a pH in the range from about 4.0 to about 6.0.

In an embodiment, the delivery vehicle comprises microparticles having same or different amounts of tretinoin, so as to modulate the release of therapeutically effective amounts of tretinoin to the application site.

In another aspect of the invention, the present invention also provides a process of preparing the substantially porous micro-particle which involves the steps of
i) dissolving tretinoin and ethyl cellulose in the organic solvent such as dichloromethane
ii) preparing an aqueous phase comprising a suspending agent
iii) adding the solution of step i) to solution of step ii under stirring or using homogenizer
iv) removing the organic solvent by stirring the emulsion, optionally, under vacuum, at a speed suitable to form the microparticles of desired porosity.

Also the present invention provides a process that does not require use of plasticizers.

The present invention also relates to a process of preparing substantially porous micro-particles wherein the process involves
i) dissolving tretinoin and ethyl cellulose in the organic solvent
ii) preparing an aqueous phase comprising a suspending agent
iii) adding the solution of step 1 to solution of step 2 under stirring or using homogenizer
iv) removing the organic solvent by stirring the emulsion, optionally under vacuum, at a speed suitable to form the microparticles of desired porosity.

The microparticles of the present invention can be prepared by any technique known in the art. Most commonly used techniques include solvent evaporation, co-acervation phase separation, spray drying, spray congealing, supercritical fluid extraction and the like.

A variety of suspending agents may be added to the solution, suspension, or emulsion during the process of microparticle preparation. The examples of cationic, anionic and nonionic compounds that may be used as suspending agents include, but are not limited to, polyvinyl alcohol (PVA), carboxymethylcellulose, polyvinyl pyrrolidone, polysorbate 80, sodium lauryl sulphate and the like. The concentration of such compounds should be sufficient to stabilize the emulsion. Polyvinyl alcohol may be present in an amount ranging from about 0.005% w/w to about 5.00% w/w, preferably from about 0.05% w/w to about 1.50% w/w, more preferably from about 0.01% w/w to about 0.5% by weight of the microparticles.

The examples of the organic solvents that may be used for the preparation of microparticles of the present invention include, but are not limited to, methylene chloride, acetone, ethyl acetate, tetrahydro furan and the like and mixtures thereof. The ratio of ethyl cellulose to solvent that may be used ranges from about 1:3 to about 1:30, preferably from about 1:7 to about 1:20, more preferably from about 1:5 to about 1:10.

In one preferred embodiment, solvent evaporation technique is used for the preparation of the microparticles. In this embodiment, a suspending agent is dissolved in aqueous solution. Tretinoin and ethyl cellulose are dissolved in an organic solvent such as dichloromethane. The organic phase containing tretinoin is added to the aqueous phase under continuous stirring at a speed ranging from about 200 rpm to 2500 rpm, preferably from 300 rpm to 2000 rpm, more preferably from 350 rpm to 1500 rpm. The organic solvent is evaporated by stirring the emulsion with or without the application of vacuum. The said emulsion stirring speed may range from about 10 rpm to 1000 rpm, preferably from about 50 rpm to 750 rpm and most preferably from 90 rpm to 500 rpm.

It was surprisingly found by the inventors that rate of evaporation of the organic solvent was found to affect the porosity of the microparticles. In one embodiment, it was found that the rate of evaporation when varied from 7.5% to 45% per hour of the total organic solvent content, for example, dichloromethane, the particles formed were substantially porous in nature. Generally, the rate of evaporation of a volatile solvent like dichloromethane is affected by the concentration of a suspending agent such as polyvinyl alcohol. At higher concentration of such suspending agents, viscosity is high and therefore, rate of evaporation of volatile solvents is low which leads to formation of microparticles with reduced porosity. It may be noted that the porous nature of the microparticles of the present invention allows higher surface area compared to the non porous microparticles. Accordingly, less drug loading is required since higher amount of drug is available to exert therapeutic effect at the site of action compared to that of the non porous microparticles. The porous nature of the particles helps in controlling the release of drug from the core matrix of the particle.

The microparticles prepared by the emulsification process may be carried out in conventional apparatus known to those skilled in the art, which include but are not limited to, static mixer, blender, magnetic bar agitation, over head stirrer, homogenizer and the like. Other apparatus conventional in the pharmaceutical art may also be employed.

The microparticles so formed may be isolated by using standard mesh sieves or by centrifugation, followed by washing with aqueous or other appropriate medium, and air dried. The isolated microparticles may be dried by application of vacuum at room temperature or by lyophilization (freeze-drying). Other collection and drying methods conventional in the pharmaceutical art may also be used.

The substantial porous nature of the microparticles of the present invention can be imagined by the electron scanning microscopic photographs as submitted herein. Also, the microparticles were subjected to in vitro dissolution test to check the release of the tretinoin from the microparticles alone. It was observed that the microparticles alone showed release of tretinoin. The applicants, without wishing to be bound by any theory, believe that it is because of the substantial porous nature of the microparticles of the present invention, the tretinoin is released because otherwise tretinoin is insoluble in water. The in vitro release of the microparticles alone is described in example 5 below. Typically, a Franz Diffusion Cell is employed however any other suitable method of determining the in vitro release profile of the microparticles may be used.

The bioequivalence of Tretinoin Microsphere gel in an aqueous gel base according to present invention, was compared to the presently FDA approved formulation (Retin A MICRO®), the SD rat model was used. Thus, topical Tretinoin produces a dose dependent reduction in the size of thickness of P. Acne induced inflamed ear. In this study, equivalent concentration of Tretinoin (0.1% w/w) in the gel formulation and the Retin A Micro® was applied to the ear of the animal for up to 15 to 19 days. At alternate days the thickness of inflamed ear was taken and the reduction in inflammation as compared to day 1 was taken to assess the effect of the Tretinoin on the reduction of P. acne induced inflammation. The resulting reduction in inflammation as described in Example 7 shows that equivalent activity in the inflammation with Tretinoin in either the formation or Retin A MICRO® formulation.

The receptor medium of the in-vitro diffusion cell comprises higher amount of volatile solvents which generally restricts the diffusion testing for only few hours rather than 24 hours. However, the in vivo efficacy testing revealed that the microparticles in release the tretinoin such that the gel can be applied once a day. This is evident from the bioequivalence determination with a FDA approved marketed topical gel available under the trade name Retin A MICRO® the results of the bioequivalence described in example 7 below.

It will be understood by those of skill in the art that numerous modifications can be made without departing from the spirit of the present invention. Therefore, it should be clearly understood that the following examples are illustrative only and should not to be construed to limit the scope of the present invention.

### Example 1

| Ingredients | Quantity (g)/batch | % w/w |
|---|---|---|
| Tretinoin | 7.50 | 1.25 |
| Ethyl Cellulose 20 cps | 582.00 | 96.75 |
| Dichloromethane | 6000.00 | — |
| Butylated Hydroxy Toluene (BHT) | 12.00 | 1.99 |
| Polyvinyl Alcohol (PVA) | 12.00 | — |
| Purified Water | 23988.00 | — |

Specified amount of tretinoin was dissolved in dichloromethane. Butylated hydroxyl toluene was added to this drug solution. Ethyl cellulose was added to the above solution and was shaken till it completely dissolves. In a separate container, polyvinyl alcohol was dissolved in specified amounts of purified water. The drug solution was added to this aqueous solution containing polyvinyl alcohol at a delivery rate of 200 ml per minute and was homogenized at 480 rpm. This mixture was stirred for 8 hours at 480 rpm under vacuum at −100 Mm Hg at 37° C. At the end of stirring, the slurry so obtained in filtered through 2-20 μm glass fiber filter paper and vacuum dried. The particle size and the specific surface area of the microparticles were determined using Malvern Mastersizer 2000. The microparticles had a particle size distribution of D₁₀<8.426 μm, D₅₀<23.353 μm and D₉₀<53.798 μm. The total porosity of the particle was determined by mercury dilatometer and was found to be 35.55%. The average pore diameter was found to be 12.601 μm. The specific surface area is 0.425 m²/g. The porous nature of the micro-particle prepared according to example 1 is illustrated in FIG. 1 which depicts the image of one of the particle under Scanning electron microscope at a magnification of 3588×).

### Example 2

| Ingredients | Quantity (g)/batch | % w/w |
|---|---|---|
| Tretinoin | 3.75 | 1.25 |
| Ethyl Cellulose 20 cps | 291.00 | 96.75 |
| Dichloromethane | 3000.00 | — |
| Butylated Hydroxy Toluene (BHT) | 6.00 | 1.99 |
| Polyvinyl Alcohol (PVA) | 6.00 | — |
| Purified Water | 11994.00 | — |

Specified amount of Tretinoin was dissolved in dichloromethane. Butylated hydroxyl toluene was added to this drug solution. Ethyl cellulose was added to the drug solution and was shaken till it completely dissolves. In a separate container, polyvinyl alcohol was dissolved in specified amounts of purified water. The drug solution was added to this aqueous solution containing polyvinyl alcohol at a delivery rate of 200 ml per minute and homogenized at 1000 rpm. This mixture was stirred for 8 hours at 1000 rpm under vacuum at −100 Mm Hg at 37° C. At the end of stirring, the slurry so obtained in filtered through 2-20 μm glass fiber filter paper and vacuum dried.

The size of the microparticles was determined using Malvern Mastersizer 2000. The microparticles had a particle size distribution of D₁₀<4.133 μm, D₅₀<15.028 μm and D₉₀<30.043 μm. The total porosity of the particle was determined by mercury dilatometer and was found to be 52.12%. The average pore diameter was found to be 3.53 μm. The specific surface area is 0.724 m²/g. The porous nature of the microparticle prepared according to example 2 is illustrated by FIG. 2 (image of one of the particle under Scanning electron microscope at a magnification of 3947×).

### Example 3

| Ingredients | Quantity (g)/batch | % w/w |
|---|---|---|
| Tretinoin | 6.25 | 1.25 |
| Ethyl Cellulose 20 cps | 485.00 | 96.75 |
| Dichloromethane | 5000.00 | — |
| Butylated Hydroxy Toluene (BHT) | 10.00 | 1.99 |
| Polyvinyl Alcohol (PVA) | 10.00 | — |
| Purified Water | 19990.00 | — |

Specified amount of Tretinoin was dissolved in dichloromethane. Butylated hydroxyl toluene was added to this drug solution. Ethyl cellulose was added to the above solution and was shaken till it completely dissolves. In a separate container, polyvinyl alcohol was dissolved in specified amounts of purified water. The drug solution was added to this aqueous solution containing polyvinyl alcohol at a delivery rate of 300 ml per minute and homogenized at 480 rpm. This mixture was stirred for 8 hours at 480 rpm under vacuum at −100 Mm Hg at 37° C. At the end of stirring, the slurry so obtained in filtered through 2-20 μm glass fiber filter paper and vacuum dried. The particle size and the specific surface area of the microparticles were determined using Malvern Mastersizer 2000. The microparticles had a particle size distribution of D₁₀<9.919 μm, D₅₀<34.35 μm and D₉₀<76.351 μm. The specific surface area is 0.334 m²/g. The porous nature of the microparticle prepared according to example 3 is illustrated in FIG. 3 (photographic image of one of the particle under Scanning electron microscope at a magnification of 3146×). The total porosity of the particle was determined by mercury dilatometer and was found to be 43.9366%. The average pore diameter was found to be 15.965 μm.

### Example 4

| Ingredient | Weight percent |
|---|---|
| Tretinoin microsphere 1% w/w of example 2 | 10.0 |
| Disodium EDTA | 0.1 |
| Sorbic acid | 0.1 |
| Glycerin | 2.5 |
| Carbopol 974P | 1.0 |
| Propylene glycol | 2.5 |
| Butylated hydroxy toluene | 0.5 |
| PPG 20 methyl glucose ether distearate | 4.75 |
| Cyclomethicone and dimethicone copolyol | 2.3 |
| Trolamine | 0.55 |
| Purified water | q.s |

Disodium EDTA and Sorbic acid was dissolved in purified water (previously heated to 60±5° C.). The solution was cooled to room temperature. Tretinoin microsphere were dispersed in the mixture of step 2 and stirred for 2 hours. Carbopol 974P was dispersed in the mixture of step 3 under stirring to obtain uniform dispersion. Glycerin was added to the mixture of step 4. Butylated hydroxy toluene was dissolved in propylene glycol under stirring (previously heated to 60±5° C.). PPG-20-methyl glucose ether distearate was added to the mixture of step 6. The mixture of step 7 was added to the dispersion of step 5 with stirring. Cyclomethicone and dimethicone copolyol was added to the mixture of step 8. Tromethamine was added to the mixture of step 9 for neutralization. Final weight adjusted with purified water. The pH of the final mixture was observed to be 5.5.

Both the Tretinoin microsphere 1% w/w and Tretinoin microspheres gel 0.1% w/w was studied for stability. It was found that the microparticles as well as the gel suspended with the microparticles shows good physical and chemical stability when stored.

### Stability Test Result for Tretinoin Microspheres 1% w/w

| | Percent degradation of Tretinoin | | |
|---|---|---|---|
| | 40° C./75% | | |
| month | RH | 30° C./65% RH | 25° C./60% RH |
| 1 M | 1.28 | 1.01 | 1.33 |
| 2 M | 1.27 | 1.17 | 1.13 |
| 3 M | 0.95 | 0.88 | 1.07 |
| 6 M | 0.54 | 0.36 | 0.35 |

### Stability Test Result for Tretinoin Microsphere Gel 0.1% w/w

| | Percent degradation of Tretinoin | | |
|---|---|---|---|
| month | 40° C./75 RH | 30° C./65 RH | 25° C./60 RH |
| 1 M | 0.38 | 0.40 | 0.42 |
| 2 M | 0.85 | 0.63 | 0.54 |
| 3 M | 1.78 | 1.81 | 1.67 |
| 6 M | 1.55 | 1.19 | 1.02 |

### Example 5

The microparticles were prepared by procedure similar to the example 2. The microparticles so prepared had an average pore diameter of 3.7174 μs with D10 4.133 μs, D50 15.028 μs and D90 30.043 μs with a specific surface area is 0.724 sq. meter/gram. These microparticles were suspended in 1% polyvinyl alcohol and subjected to the in vitro dissolution testing. In vitro release method uses an open chamber diffusion cell system such as a Franz cell system, fitted usually with a synthetic membrane. The suspended microparticles sample is placed on the upper side of the membrane in the open donor chamber of the diffusion cell and a sampling fluid is placed on the other side of the membrane in a receptor cell. Diffusion of drug from the topical product to and across the membrane is monitored by assay of sequentially collected samples of the receptor fluid. Aliquots removed from the receptor phase can be analyzed for drug content by high pressure liquid chromatography (HPLC) or other analytical methodology. A plot of the amount of drug released per unit area (mcg/cm2) against the square root of time yields a straight line, the slope of which represents the release rate. The membrane system is Supor 450, 0.45μ pore size, 47 mm diameter with a receptor phase having a 50% Isopropyl alcohol (IPA) solution with 1% Butylated Hydroxy Toluene (BHT) was used as the receptor phase. The in-vitro release of tretinoin from the microparticles alone and from the gel in which microparticles are suspended is as below:

| | % tretinoin released from the |
|---|---|
| Time in hours | microparticles |
| 1 | 1.81 |
| 2 | 3.36 |
| 3 | 9.32 |
| 5 | 15.53 |
| 7 | 22.98 |

### Example 6

In order to assess the bioequivalence of Tretinoin Microsphere gel in an aqueous gel base according to present invention, compared to the presently FDA approved formulation (RETIN A MICRO®), the SD rat model was used. Thus, topical Tretinoin produces a dose dependent reduction in the size of thickness of P. acne induced inflamed ear. In this study, equivalent concentration of Tretinoin (0.1% w/w) in the gel formulation and the Retin A Micro® was applied to the ear of the animal for up to 15 to 19 days. At alternate days the thickness of inflamed ear was taken and the reduction in inflammation as compared to day 1 was taken to assess the effect of the Tretinoin on the reduction of P. acne induced inflammation. The resulting reduction in inflammation shows that equivalent activity in the inflammation with Tretinoin in either the formation or Retin A Micro® formulation.

### Comparative Efficacy Study of Tretinoin Microsphere Gel 0.1% w/w vs Retin A Micro®

% change as compared to day 1

| Day | Tretinoin microsphere gel | Retin A Micro |
|---|---|---|
| 3 | 21.52 | 10.44 |
| 5 | 19.24 | 16.27 |
| 7 | 6.1 | 23.09 |
| 9 | 18.29 | 10.64 |
| 11 | 7.62 | 5.22 |
| 13 | −18.29 | −11.45 |
| 15 | −28.02 | −25.7 |
| 17 | −18.86 | −17.07 |
| 19 | −23.05 | −15.46 |

### 2. Comparative Efficacy Study of Tretinoin Microsphere Gel 0.1% w/w vs Retin A Micro® % Change in Inflammation as Compared to Day 1

% change as compared to day 1

| | Tretinoin microsphere gel | Retin A Micro ®- |
|---|---|---|
| Day | of present invention | marketed product |
| 3 | 18.44 | 15.86 |
| 5 | 19.44 | 8.51 |
| 7 | 9.62 | 1.16 |
| 9 | 7.41 | −1.16 |
| 11 | 0.40 | −7.35 |
| 13 | −4.01 | −9.28 |
| 15 | −15.43 | −16.05 |

### Example 7

To assess the dermal irritation, after single dose of tretinoin microsphere gel and its placebo was applied on NZW rabbit. 0.5 ml of tretinoin microsphere gel and its placebo were applied on approximately 6 cm² of right and left dorso lateral areas respectively and covered with a gauge patch and non irritating tape. After 4 hours of application gauge was removed and residual test substance was washed with saline. All animals were examined for the signs of erythema, eschar/oedema and responses were scored at 1, 24, 48, 72 hours after patch removal.

### Skin Irritation Scores to the Grades

Erythema and Eschar formation (Maximum possible 4)

| | |
|---|---|
| No Erythema | 0 |
| Very slightly erythema (barely perceptible0 | 1 |
| Well defined Erythema | 2 |
| Moderate to severe Erythema | 3 |
| Severe erythema (beef redness) to Eschar formation preventing | 4 |
| grading of erythema | |

Oedema Formation (Maximum possible 4)

| | |
|---|---|
| No Oedema | 0 |
| Very slight oedema (barely perceptible) | 1 |
| Slight oedema (edges of area well defined by definite raising) | 2 |
| Moderate oedema (Raised approximately 1 millimeter) | 3 |
| Severe Oedema (Raised more than 1 millimeter snd extending | 4 |
| beyond area of exposure) | |

| | | Time | Erythema and | | | | | |
|---|---|---|---|---|---|---|---|---|
| Rabbit | | point | Eschar formation | | Edema formation | | Necrosis | |
| numbers | Details | (hrs) | Right | Left | Right | Left | Right | Left |
| 1 | Right side: | 24 | 0 | 0 | 0 | 0 | NAD | NAD |
| | 0.5 ml | 48 | 0 | 0 | 0 | 0 | NAD | NAD |
| | Tretinoin | 72 | 0 | 0 | 0 | 0 | NAD | NAD |
| | microsphere | Total | 0 | 0 | 0 | 0 | NAD | NAD |
| 2 | Gel 0.1% | 24 | 0 | 0 | 0 | 0 | NAD | NAD |
| | w/w applied | 48 | 0 | 0 | 0 | 0 | NAD | NAD |
| | Left side: | 72 | 0 | 0 | 0 | 0 | NAD | NAD |
| | 0.5 ml | Total | 0 | 0 | 0 | 0 | NAD | NAD |
| 3 | placebo | 24 | 0 | 0 | 0 | 0 | NAD | NAD |
| | applied | 48 | 0 | 0 | 0 | 0 | NAD | NAD |
| | | 72 | 0 | 0 | 0 | 0 | NAD | NAD |
| | | Total | 0 | 0 | 0 | 0 | NAD | NAD |

No tissue reaction in the form of erythema, edema, or necrosis was observed in tretinoin microsphere gel and its placebo in application sites at any scoring intervals.

To evaluate the irritation potential by ocular route, a single dose instillation of tretinoin microsphere gel and its placebo to eyes of NZW rabbits. 0.1 ml of tretinoin microsphere gel and its placebo was instilled into right eye and left eye respectively of each rabbit. For instillation, lower eyelid was gently held together for a few seconds to prevent loss of gel. After 1 hour residual gel was washed with normal saline from eye. Rabbits were examined immediately after washing (1 hour) and at 24, 48 and 72 hours post instillation, to note signs of the eye irritation, if any.

### Eye Irritation Score to the Grades

Corneal opacity: degree of density (The area of corneal opacity)
(Maximum possible score 4)

| | |
|---|---|
| No ulceration or opacity | 0 |
| Scattered or diffuse areas of opacity, details of iris clearly visible | 1 |
| Easily discernible translucent area, detils of iris slightly obscured | 2 |
| Nacreous area, no details of iris visible, size of pupil barely | 3 |
| discernible | |
| Opaque cornea, iris not discernible through the opacity | 4 |

Iris (Maximum possible score 2)

| | |
|---|---|
| Normal | 0 |
| Markedly deeped rugae, congestion, swelling, moderate circum | 1 |
| corneal hyperaemia, or injection, any of these or combination of | |
| any thereof it is still reacting to light | |
| No reaction to light, haemorrhage, gross destruction (any or all of | 2 |
| these | |

Conjunctivae (Maximum possible score 3)

| | |
|---|---|
| Redness (refers to palpebral and bulbar conjunctivae, cornea and iris | |
| Normal | 0 |
| Some blood vessels definitely hyperaemic (injected) | 1 |
| Diffuse, crimson colour, individual vessels not easily discernible | 2 |
| Diffuse beefy red | 3 |

Chemosis: swelling (refers to lids and/or nictiating membranes)
maximum possible score 4

| | |
|---|---|
| Normal | 0 |
| Some swelling above normal | 1 |
| Obvious swelling with partial eversion of lids | 2 |
| Swelling with lids about half closed | 3 |
| Swelling with lids more than half closed | 4 |

In ⅓ rabbit some blood vessels definitely hyperemic in conjuctivae observed at 1 hour in right eye and gets disappeared after 24 hours. In ⅓ rabbit blood vessels of conjuctvae not easily discernible at 1 hour which gets disappeared at 48 hours. No abnormality was noticed in placebo instilled left eye of any rabbit. No iris or cornea involvement was observed in any of the animal.

1. A substantially porous, micro-particle comprising therapeutically effective amounts of tretinoin and ethyl cellulose.

2. A substantially porous micro-particles as claimed in claim 1 wherein the micro-particles are free of plasticizers.

3. A substantially porous micro-particles as claimed in claim 1 wherein the average pore diameter of the micro-particles ranges from about 2 microns to 20 microns.

4. A substantially porous, micro-particle as claimed in claim 1 wherein the ratio of ethyl cellulose to tretinoin ranges from about 99.0:1.0 to about 50:50.

5. A substantially porous, micro-particle as claimed in claim 1 wherein tretinoin is loaded to about 1% by weight.

6. A substantially, porous micro-particle as claimed in claim 1 wherein the pores are formed in situ by use of volatile solvent such as dichloromethane.

7. A process of preparing the substantially porous micro-particle which involves the steps of
i) dissolving tretinoin and ethyl cellulose in the organic solvent such as dichloromethane
ii) preparing an aqueous phase comprising a suspending agent
iii) adding the solution of step i) to solution of step ii under stirring or using homogenizer
iv) removing the organic solvent by stirring the emulsion, optionally, under vacuum, at a speed suitable to form the microparticles of desired porosity.

8. A process as claimed in claim 7 wherein the process does not require use of plasticizers.

9. A process as claimed in claim 8 wherein the amount of suspending agent ranges from about 0.01% w/w to about 0.5% by weight of the microparticles.

10. A process as claimed in claim 7 wherein the rate of removal of organic solvent ranges from 7.5% to 45% per hour of the total organic solvent content.

11. A process as claimed in claim 7 wherein the stirring speed during removal of organic solvent ranges from 350 rpm to 1500 rpm.
